(19) United States
(12) Patent Application Publication
(10) Pub. No.: US 2020/0172924 A1
(43) Pub. Date: Jun. 4, 2020

(54) EXPERIMENTAL RESEARCH METHOD FOR TARGETED THERAPY OF PROSTATE CANCER BY NUCLIDE 125I-LABELED DUAL-REGULATION ONCOLYTIC ADENOVIRUS

(71) Applicant: Conghui Han, Jiangsu (CN)

(72) Inventors: Conghui Han, Jiangsu (CN); Lin Hao, Jiangsu (CN); Zhenduo Shi, Jiangsu (CN); Xiaoxiao Han, Shanghai (CN)

(73) Assignee: Conghui Han, Jiangsu (CN)

(21) Appl. No.: 16/392,607

(22) Filed: Apr. 23, 2019

(30) Foreign Application Priority Data
Dec. 4, 2018 (CN) 201811476838.2

(51) Int. Cl. C12N 15/85

(52) U.S. Cl. CPC C12N 15/8509; C12N 2830/008; C12N 2015/8572

(57) ABSTRACT

The invention provides an experimental research method for targeted therapy of prostate cancer by nuclide ¹²⁵I-labeled dual-regulation oncolytic adenovirus, comprising cloning and identification of prostate specific antigen PSA promoter; of adenoid construction and identification of hTERT/PSA double-regulated adenovirus vector RSOAds-hTERT/PSA; nuclide ¹²⁵I-labeled hTERT/PSA dual-regulation proliferative oncolytic adenovirus construction ¹²⁵I-RSOAds-hTERT/PSA nuclide-oncolytic virus marker; detection of transfection efficiency and tumor killing effect of ¹²⁵I-RSOAds-hTERT/PSA on hormone-independent prostate cancer cells in vitro; ¹²⁵I-RSOAds-hTERT/PSA targeted therapy for anti-tumor effect of prostate cancer and observation of tumor microenvironment changes. The invention can realize accurate impact analysis, and realizes the detection and analysis of the effect of the dual-regulated oncolytic adenovirus of the ¹²⁵I-labeled PSA/hTERT promoter on prostate cancer targeted therapy and tumor microenvironment through a comprehensive experimental method, and ensures the accuracy and reliability of the results.

### CROSS-REFERENCE TO RELATED APPLICATION

This application claims the priority benefit of China application serial no. 201811476838.2, filed on Dec. 4, 2018. The entirety of the above-mentioned patent application is hereby incorporated by reference herein and made a part of this specification.

### BACKGROUND

### Technical Field

The invention relates to a method for labeling double-regulating oncolytic adenovirus with 125I labeled by radionuclide and an experimental research method for targeted therapy of prostate cancer.

### Description of Related Art

Prostate cancer refers to an epithelial malignancy that occurs in the prostate. In 2004, WHO pathology and genetics of urinary system and male reproductive organ tumors included adenocarcinoma (adenocarcinoma), ductal adenocarcinoma, urothelial carcinoma, squamous cell carcinoma, and adenosquamous carcinoma. Among them, prostate adenocarcinoma accounts for more than 95%. Therefore, prostate cancer is usually referred to as prostate adenocarcinoma. In 2012, the incidence rate of prostate cancer in the cancer registration area in China was 9.92/100,000, ranking the sixth in the incidence of male malignant tumors.

The research of prostate cancer is one of the important research topics. At present, there is no accurate study on the effect of dual-regulated oncolytic adenovirus with the 125I-labeled PSA/hTERT promoter on prostate cancer targeted therapy and tumor microenvironment. Nuclide 125I-labeled PSA/hTERT promoter dual-regulation of oncolytic adenovirus, this method has rarely been reported at home and abroad.

### SUMMARY

The object of the present invention is to provide a method for labeling dual-regulating oncolytic adenovirus with 125I and its experimental research method for targeted therapy of prostate cancer, and solving the problem of how to carry out the radionuclide 125I-labeled PSA/hTERT promoter in the prior art. The problem of regulating the accurate study of the effect of oncolytic adenovirus on prostate cancer targeted therapy and tumor microenvironment.

The technical solution of the present invention is:

A method for labeling dual-regulating oncolytic adenovirus with 125I labeled nuclide and an experimental research method thereof for targeted therapy of prostate cancer, including the following steps

S1, cloning and identification of prostate specific antigen PSA promoter;

S2, Construction and identification of S2, hTERT/PSA double-regulated adenovirus vector RSOAds-hTERT/PSA;

S3, nuclide 125I-labeled hTERT/PSA dual-regulation proliferative oncolytic adenovirus construct 125I-RSOAds-hTERT/PSA nuclide-oncolytic virus marker;

S4, Detection of transfection efficiency and tumor killing effect of S4, 125I-RSOAds-hTERT/PSA on hormone-independent prostate cancer cells in vitro;

S5, 125I-RSOAds-hTERT/PSA targeted therapy for anti-tumor effect of prostate cancer, observation of tumor microenvironment changes.

Further, step S1 is specifically

S11, gene-cloning and identification of prostate-specific promoter: According to the sequence obtained by NCBI Nucleotide (U37672.1), the PSA promoter primers were designed and amplified, and the NotI and SpeI sites were introduced respectively; from prostate cancer tissue the genomic DNA was extracted and the 522 bp PSA promoter fragment was amplified by PCR. The pUC57 plasmid was digested with EcoR V, and the PCR product of the target gene was ligated with the pUC57 vector, and the plasmid was isolated and cloned. And sequencing confirmed the insertion product; the correct plasmid was named pUC57-PSAp;

S12, PSA promoter and hTERT promoter biological activity assay: cultured prostate cancer cells expressing different PSA and hTERT to logarithmic growth phase, transfected with luciferase plasmids PGL3-PSA and hTERT containing PSA promoter and hTERT promoter. The dual luciferase system measures the biological activity of the PSA promoter and the hTERT promoter.

Further, step S2 is specifically:

S21, Construction of hTERT/PSA dual-regulated adenoviral vector RSOAds-hTERT/PSA: deletion of the E1A and E1B promoters of adenoviral plasmid PQW1 by site-directed mutagenesis polymerase chain reaction and production of appropriate restriction sites. The hTERT promoter and the PSA promoter with the same restriction enzyme site were ligated into the PQW1 vector to obtain a double-regulated proliferation adenovirus vector RSOAds-hTERT/PSA;

S22. Each group of viruses was subjected to amplification and titer determination.

Further, step S3 is specifically:

S31, The 125I label was labeled with N-bromosuccinimide (NBS) as an oxidant, and different concentrations of oncolytic virus and NBS were set to determine the optimal labeling conditions. The amount of 125I, reaction time, The effect of pH and reaction volume on the labeling rate of 125I-RSOAds-hTERT/PSA nuclides-oncolytic virus markers;

S32, After the labeling was completed, the nuclear-oncolytic adenovirus marker was isolated and purified by gel column chromatography; the radioactivity of 125I-RSOAds-hTERT/PSA label was determined by paper chromatography at different times and filtered by microfiltration membrane. Bacteria, and put the markers in a 4° C. refrigerator for use;

S33, 125I was labeled with dual-regulated oncolytic adenovirus to complete the pre-experiment, and the optimal labeling method, labeling conditions, labeling rate, and external conditions including temperature, time and pH were used to influence the success of the labeling.

Further, step S4 is specifically:

S41, Two kinds of prostate cancer cell lines including human androgen-independent prostate cancer cell line PC3, mouse androgen-independent prostate adenocarcinoma cell line RM-1, and normal prostate tissues were determined by RT-PCR and Western blot, respectively. hTERT/PSA biological activity expression;

S42, The prostate cancer cells with different expression levels of hTERT/PSA were cultured, and the expression of surface molecules such as prostate cancer stem cell antigens PSCN, CD44+ and CD24+ in each group were detected.

S43, In vitro experiments were divided into 4 groups: virus-nuclear complex group (125I-RSOAds-hTERT-PSA), non-nuclear-labeled RSOAds-hTERT-PSA group, simple nuclear group 125I group, normal saline blank control group, timely according to experimental requirements. Two types of in vitro cultured prostate cancer cells were added to each experimental group, and the killing effects of the above four groups on prostate cancer cells with different hTERT and PSA expression were compared.

S44, Each group was tested as follows and repeated at least 3 times. The collected data were subjected to detailed statistical analysis to observe the killing effect of 125I-RSOAds-hTERT/PSA on prostate cancer cell growth:

\textcircled{1} Detection of oncolytic adenovirus E1A/E1B gene expression in 125I-RSOAds-hTERT/PSA; \textcircled{2} Detection of oncolytic adenovirus replication ability in 125I-RSOAds-hTERT/PSA; \textcircled{3} Determination of the killing effect of 125I-RSOAds-hTERT/PSA on prostate cancer cells; \textcircled{4} ELISA was used to detect the secretion of cytokines in the supernatant of each group, and to understand the changes of immune indicators; \textcircled{5} TUNNEL and flow cytometry were used to detect the apoptosis of prostate cancer cells in each group, and some special apoptosis-inducing indicators were detected; \textcircled{6} Expression of prostate cancer stem cell antigens PSCN, CD44+, CD24+ on prostate cancer tumor cells;

S45, The concentration of radionuclide 125I in prostate cancer cells was detected. The equivalent killing effect was measured by dose of nuclides 125I, and the stability of nuclides 125I was detected in different culture periods.

Further, step S5 is specifically

S51, Distribution of radionuclide-virus complexes in normal mice: 125I-RSOAds-hTERT-PSA was injected from the tail vein of mice, and ECT or PET-CT imaging was used at different time intervals to determine the standard maximum uptake value of different organ nuclide;

S52, Establishing an animal model of implanted inbred C57BL/6 mouse prostate cancer with reference to international and domestic literature: preparing a cell suspension of mouse androgen-independent prostate adenocarcinoma cell line RM-1 in log phase in vitro, and 10×106/mouse was injected subcutaneously into the right forelimb of the mouse or other suitable sites, and the tumor formation was observed by micro-ultrasound and touch method. The tumor was observed at about 2 g, and the blood was passed through the tail vein. PSA and some cytokines;

S53, Animal experiments were randomly divided into 4 groups (n=20): radionuclide-virus (125I-RSOAds-hTERT-PSA) marker group, unlabeled radionuclide RSOAds-hTERT-PSA group, simple radionuclide group 125I, saline blank control In the group, mice in each experimental group were treated with direct injection of prostate cancer and intravenous administration of mice, and the 125I-RSOAds-hTERT-PSA marker, dual-regulated oncolytic adenovirus RSOAds-hTERT-PSA, and simple Application of anti-tumor effect of radionuclide 125I;

S54, Each experimental group was observed and tested as follows in different time periods:

\textcircled{1} Subcutaneous tumor growth curve, survival observation and volume of transplanted tumor in tumor-bearing mice (using micro-ultrasound); \textcircled{2} The protein content of transfected adenovirus E1A/E1B was detected in tumor tissues, and the transfection efficiency was studied. It was observed whether 125I-RSOAds-hTERT-PSA can directly target prostate cancer cells; \textcircled{3} Prostate cancer cell apoptosis detection (TUNNEL method and flow cytometry); Western Blot was used to detect the expression level of Caspase-3 and explore the apoptosis-inducing pathway; \textcircled{4} Heterotopic transplantation of prostate cancer and pathological examination of important organs (HE staining, immunohistochemistry), examination of CD4+, CD8+ T cells and macrophage infiltration in the tumor; \textcircled{5} ELISA method was used to detect the secretion of cytokines in mouse serum including IL-2, TNF, IL-10 and IFN-γ; 6PSA changes.

S55, The microinvasiveness and microangiogenesis of implanted prostate tumor tissues were observed by electron microscopy. The expressions of VGEF, PSCN, CD44+, CD31+ and C D24+ in tumor tissues were detected. The infiltration of inflammatory cells in pathological specimens was detected. Environmental change;

S56, ECT or PET-CT imaging method was used to observe the distribution of 125I in mice after treatment. The toxic side effects of 125I-RSOAds-hTERT/PSA in tumor-bearing mice were observed and the dosage was applied.

The beneficial effects of the invention are as follows: the method for labeling dual-regulating oncolytic adenovirus with 125I labeled by radionuclide and its experimental research method for targeted therapy of prostate cancer can realize accurate impact analysis, and realize through comprehensive experimental methods. The detection of the dual-regulated oncolytic adenovirus of the 125I-labeled PSA/hTERT promoter on prostate cancer targeted therapy and tumor microenvironment ensures the accuracy and reliability of the results.

### DESCRIPTION OF THE EMBODIMENTS

### Example

A method for labeling dual-regulating oncolytic adenovirus with 125I labeled nuclide and an experimental research method thereof for targeted therapy of prostate cancer, including the following steps

S1 Cloning and identification of prostate specific antigen PSA promoter;

Step S1 is specifically

S11, PCR cloning and identification of prostate-specific promoters: According to the sequence obtained by NCBI Nucleotide (U37672.1), the PSA promoter primers were designed and amplified, and the NotI and SpeI restriction sites were introduced respectively. Extracted from prostate cancer tissues. The 522 bp PSA promoter fragment was amplified by genomic DNA, and the pUC57 plasmid was digested with EcoR V. The PCR product of the target gene was ligated with the pUC57 vector, the clone was selected, the plasmid was extracted and sequenced. Confirm the insertion product; name the correct plasmid as pUC57-PSAp;

S12, PSA promoter and hTERT promoter biological activity assay: cultured prostate cancer cells expressing different PSA and hTERT to logarithmic growth phase, transfected with luciferase plasmids PGL3-PSA and hTERT containing PSA promoter and hTERT promoter. The dual luciferase system measures the biological activity of the PSA promoter and the hTERT promoter.

S2, Construction and identification of hTERT/PSA dual-regulated adenovirus vector RSOAds-hTERT/PSA;

Step S2 is specifically

S21, Construction of hTERT/PSA dual-regulated adenoviral vector RSOAds-hTERT/PSA: deletion of the E1A and E1B promoters of adenoviral plasmid PQW1 by site-directed mutagenesis polymerase chain reaction and production of appropriate restriction sites. The hTERT promoter and the PSA promoter with the same restriction enzyme site were ligated into the PQW1 vector to obtain a double-regulated proliferation adenovirus vector RSOAds-hTERT/PSA;

S22, Amplification and titer determination were performed for each group of viruses.

S3, Construction of 125I-RSOAds-hTERT/PSA nuclide-oncolytic virus marker by radionuclide 125I-labeled hTERT/PSA dual-regulated proliferative oncolytic adenovirus;

Step S3 is specifically

S31, The 125I label was labeled with N-bromosuccinimide (NBS) as an oxidant, and different concentrations of oncolytic virus and NBS were set to determine the optimal labeling conditions. The amount of 125I, reaction time, The effect of pH and reaction volume on the labeling rate of 125I-RSOAds-hTERT/PSA nuclides-oncolytic virus markers;

S32, After the labeling was completed, the nuclear-oncolytic adenovirus marker was isolated and purified by gel column chromatography; the radioactivity of 125I-RSOAds-hTERT/PSA label was determined by paper chromatography at different times and filtered by microfiltration membrane. Bacteria, and put the markers in a 4° C. refrigerator for use;

S33, 125I was labeled with dual-regulated oncolytic adenovirus to complete the pre-experiment, and the optimal labeling method, labeling conditions, labeling rate, and external conditions including temperature, time and pH were used to influence the success of the labeling.

S4, Detection of transfection efficiency and tumor killing effect of 125I-RSOAds-hTERT/PSA on hormone-independent prostate cancer cells in vitro;

Step S4 is specifically

S41, Two kinds of prostate cancer cell lines including human androgen-independent prostate cancer cell line PC3, mouse androgen-independent prostate adenocarcinoma cell line RM-1, and normal prostate tissues were determined by RT-PCR and Western blot, respectively. hTERT/PSA biological activity expression;

S42, The prostate cancer cells with different expression levels of hTERT/PSA were cultured, and the expression of surface molecules such as prostate cancer stem cell antigens PSCN, CD44+ and CD24+ in each group were detected.

S43, In vitro experiments were divided into 4 groups: virus-nuclear complex group (125I-RSOAds-hTERT-PSA), non-nuclear-labeled RSOAds-hTERT-PSA group, simple nuclear group 125I group, normal saline blank control group, timely according to experimental requirements. Two types of in vitro cultured prostate cancer cells were added to each experimental group, and the killing effects of the above four groups on prostate cancer cells with different hTERT and PSA expression were compared.

S44, Each group was tested as follows and repeated at least 3 times. The collected data were subjected to detailed statistical analysis to observe the killing effect of 125I-RSOAds-hTERT/PSA on prostate cancer cell growth:

\textcircled{1} Detection of oncolytic adenovirus E1A/E1B gene expression in 125I-RSOAds-hTERT/PSA; \textcircled{2} Detection of oncolytic adenovirus replication ability in 125I-RSOAds-hTERT/PSA; \textcircled{3} The killing effect of 125I-RSOAds-hTERT/PSA on prostate cancer cells was determined. \textcircled{4} The secretion of cytokines in the supernatant of each group was detected by ELISA, and the changes of immune indexes were observed. \textcircled{5} TUNNEL and flow cytometry were used to detect the apoptosis of prostate cancer cells in each group, and some specific apoptosis-inducing indicators were detected. 6 The expressions of prostate stem cell antigens PSCN, CD44+ and CD24+ on prostate cancer cells were observed.

Step S5 is specifically

S51, Distribution of radionuclide-virus complexes in normal mice: 125I-RSOAds-hTERT-PSA was injected from the tail vein of mice, and ECT or PET-CT imaging was used at different time intervals to determine the standard maximum uptake value of different organ nuclide;

S52, Establishing an animal model of implanted inbred C57BL/6 mouse prostate cancer with reference to international and domestic literature: preparing a cell suspension of mouse androgen-independent prostate adenocarcinoma cell line RM-1 in log phase in vitro, and 10×106/mouse was injected subcutaneously into the right forelimb of the mouse or other suitable sites, and the tumor formation was observed by micro-ultrasound and touch method. The tumor was observed at about 2 g, and the blood was passed through the tail vein. PSA and some cytokines;

S53, Animal experiments were randomly divided into 4 groups (n=20): radionuclide-virus (125I-RSOAds-hTERT-PSA) marker group, unlabeled radionuclide RSOAds-hTERT-PSA group, simple radionuclide group 125I, saline blank control In the group, mice in each experimental group were treated with direct injection of prostate cancer and intravenous administration of mice, and the 125I-RSOAds-hTERT-PSA marker, dual-regulated oncolytic adenovirus RSOAds-hTERT-PSA, and simple Application of anti-tumor effect of radionuclide 125I;

S54, Each experimental group was observed and tested as follows in different time periods:

\textcircled{1} Subcutaneous tumor growth curve, survival observation and volume of transplanted tumor in tumor-bearing mice (using micro-ultrasound); \textcircled{2} The protein content of transfected adenovirus E1A/E1B was detected in tumor tissues, and the transfection efficiency was studied. It was observed whether 125I-RSOAds-hTERT-PSA can directly target prostate cancer cells; \textcircled{3} Prostate cancer cell apoptosis detection (TUNNEL method and flow cytometry); Western Blot was used to detect the expression level of Caspase-3 and explore the apoptosis-inducing pathway; \textcircled{4} Heterotopic transplantation of prostate cancer and pathological examination of important organs (HE staining, immunohistochemistry), examination of CD4+, CD8+ T cells and macrophage infiltration in the tumor; \textcircled{5} ELISA method was used to detect the secretion of cytokines in mouse serum including IL-2, TNF, IL-10 and IFN-γ; 6PSA changes.

S55, The microinvasiveness and microangiogenesis of implanted prostate tumor tissues were observed by electron microscopy. The expressions of VGEF, PSCN, CD44+, CD31+ and C D24+ in tumor tissues were detected. The infiltration of inflammatory cells in pathological specimens was detected. Environmental change;

S56, ECT or PET-CT imaging method was used to observe the distribution of 125I in mice after treatment. The toxic side effects of 125I-RSOAds-hTERT/PSA in tumor-bearing mice were observed and the dosage was applied.

The method for labeling dual-regulated oncolytic adenovirus with 125I label and its experimental research method for targeted therapy of prostate cancer can achieve accurate impact analysis, and achieve a radionuclide 125I-labeled PSA/through a more comprehensive experimental method. The hTERT promoter double-regulated oncolytic adenovirus detects prostate cancer targeted therapy and tumor microenvironment, ensuring the accuracy and reliability of the results.

What is claimed is:

1. An experimental research method for targeted therapy of prostate cancer by nuclide ¹²⁵I-labeled dual-regulation oncolytic adenovirus, comprising the following steps,
S1, cloning and identification of prostate specific antigen PSA promoter;
S2, construction and identification of hTERT/PSA double-regulated adenovirus vector RS OAds-hTERT/PSA;
S3, nuclide ¹²⁵I-labeled hTERT/PSA dual-regulation proliferative oncolytic adenovirus construct ¹²⁵I-RSOAds-hTERT/PSA nuclide-oncolytic virus marker;
S4, detection of transfection efficiency and tumor killing effect of ¹²⁵I-RSOAds-hTERT/PSA on hormone-independent prostate cancer cells in vitro; and
S5, ¹²⁵I-RSOAds-hTERT/PSA targeted therapy for anti-tumor effect of prostate cancer, observation of tumor microenvironment changes.

2. The experimental research method according to claim 1, wherein the step S1 specifically comprises the following steps,
S11, gene-cloning and identification of prostate-specific promoter: according to the sequence obtained by NCBI Nucleotide (U37672.1), the PSA promoter primers were designed and amplified, and the NotI and SpeI sites were introduced respectively; from prostate cancer tissue genomic DNA was extracted and a 522 bp PSA promoter fragment was amplified by PCR; pUC57 vector was digested with EcoR V, a PCR product of the target gene was ligated with the pUC57 vector, clones were screened and the plasmid was isolated for sequencing confirmed the insertion product; the correct plasmid was named pUC57-PSAp; and
S12, PSA promoter and hTERT promoter biological activity assay: cultured prostate cancer cells expressing different PSA and hTERT to logarithmic growth phase, transfected with luciferase plasmids PGL3-PSA and hTERT containing PSA promoter and hTERT promoter, and measured the biological activity of the PSA promoter and the hTERT promoter by dual luciferase system.

3. The experimental research method according to claim 1, wherein the step S2 specifically comprises the following steps,
S21, construction of hTERT/PSA dual-regulated adenoviral vector RSOAds-hTERT/PSA: deletion of the E1A and E1B promoters of adenoviral vector PQW1 by site-directed mutagenesis polymerase chain reaction and production of appropriate restriction sites; the hTERT promoter and the PSA promoter with the same restriction enzyme site were ligated into the PQW1 vector to obtain a double-regulated proliferation adenovirus vector RSOAds-hTERT/PSA; and
S22, each group of viruses was subjected to amplification and titer determination.

4. The experimental research method according to claim 1, wherein the step S3 specifically comprises the following steps,
S31, the ¹²⁵I label was labeled with N-bromosuccinimide (NBS) as an oxidant, and different concentrations of oncolytic virus and NBS were set to determine the optimal labeling conditions; the effect of the amount of ¹²⁵I, reaction time, pH and reaction volume on the labeling rate of ¹²⁵I-RSOAds-hTERT/PSA nuclides-oncolytic virus markers were investigated;
S32, after separation, the gel-column chromatography was used to separate and purify the radionuclide-oncolytic adenovirus marker; the radiochromic purity of ¹²⁵I-RSOAds-hTERT/PSA label was determined by paper chromatography at different times, using microporous membrane filter and sterilize, and storing the marker in a 4° C. refrigerator for use; and
S33, ¹²⁵I was labeled with dual-regulated oncolytic adenovirus to complete the pre-experiment, and the optimal labeling method, labeling conditions, labeling rate, and external conditions comprising temperature, time and pH were used to influence the success of the labeling.

5. The experimental research method according to claim 1, wherein the step S4 specifically comprises the following steps,
S41, RT-PCR and Western blotting were used to identify two prostate cancer cell lines, comprising the human androgen-independent prostate cancer cell line PC3, the mouse androgen-independent prostate cancer cell line RM-1 and normal prostate tissue. Biological activity expression of hTERT/PSA;
S42, cultivating various types of prostate cancer cells with different expression levels of hTERT/PSA, and detecting expression of surface molecules such as prostate cancer stem cell antigens PSCN, CD44+, CD24+ in each group of cells;
S43, in vitro experiments were divided into 4 groups: virus-nuclear complex group (125I-RSOAds-hTERT-PSA), nuclear-free RSOAds-hTERT-PSA group, simple radionuclide group 125I, saline blank control group, according to the experiment requires the addition of two types of in vitro cultured prostate cancer cells in each experimental group, and compares the killing effects of the above four groups on prostate cancer cells with different hTERT and PSA expression;
S44, each group was tested as follows and repeated at least 3 times: a detailed statistical analysis of the collected data was performed to observe the killing effect of 125I-RSOAds-hTERT/PSA on prostate cancer cell growth:
detection of the expression of the oncolytic adenovirus E1A/E1B gene in ¹²⁵I-RSOAds-hTERT/PSA; detection of oncolytic adenovirus replication in ¹²⁵I-RSOAds-hTERT/PSA; determination of ¹²⁵I-RSOAds-hTERT/PSA on prostate cancer cells killing effect; ELISA was used to detect the secretion of cytokines in the supernatant of each group, and the changes of immune indexes were observed; the apoptosis of prostate cancer cells in each group was detected by TUNNEL method and flow cytometry, and some specific apoptosis induction index was detected; expression of prostate cancer stem cell antigens PSCN, CD44+, CD24+ in prostate cancer tumor cells was detected; and
S45, the concentration of nuclide ¹²⁵I in prostate cancer cells was examined; the dose of nuclide ¹²⁵I was measured by the same killing effect, and the stability of nuclide ¹²⁵I was examined during different culture periods.

6. The experimental research method according to claim 1, wherein the step S5 specifically comprises the following steps,
S51, distribution of nuclear-virus complexes in normal mice: ¹²⁵I-RSOAds-hTERT-PSA was injected from the tail vein of mice, and ECT or PET-CT imaging was used at different time periods to determine the standard maximum intake value of different organ nuclide;
S52, establishing an implanted inbred C57BL/6 mouse prostate cancer animal model with reference to international and domestic literature: preparing a cell suspension of mouse androgen-independent prostate adenocarcinoma cell line RM-1 in log phase in vitro, and 10×10⁶ cells/mouse was injected subcutaneously into the right forelimb of the mouse or other suitable parts; the microscopic ultrasound and the touch method were used to observe the tumor formation; the experiment was performed when the tumor was about 2 g; PSA and some cytokines were examined by blood sampling from the tail vein;
S53, animal experiments were randomly divided into 4 groups (n=20): radionuclide-virus (¹²⁵I-RSOAds-hTERT-PSA) marker group, unlabeled radionuclide RSOAds-hTERT-PSA group, simple nuclear group ¹²⁵I group, normal saline in the blank control group; mice in each experimental group were treated with direct injection of prostate cancer and intravenous administration of mice, and anti-tumor effect of the ¹²⁵I-RSOAds-hTERT-PSA marker, the double-regulated oncolytic adenovirus RSOAds-hTERT-PSA, and the application of radionuclide ¹²⁵I were compared;
S54, each experimental group performs the following observation and detection in different time periods:
1) tumor growth curve, survival observation and transplanted tumor volume of tumor-bearing mice (using micro-ultrasound); 2) detection of tumor tissue transfected adenovirus E1A/E1B protein content, study transfection efficiency, observe ¹²⁵I-RSOAds-hTERT-PSA can directly target prostate cancer cells; 3) prostate cancer cell apoptosis detection (TUNNEL method and flow cytometry); Western Blot detection of Caspase-3 and other expression levels, to explore apoptosis-induced pathways; 4) prostate cancer transplanted tumors and pathological examination of important organs (HE staining, immunohistochemistry), examination of CD4+, CD8+ T cells and macrophage infiltration in the tumor; 5) ELISA method to detect the secretion of cytokines in the serum of mice comprising IL-2, TNF, IL-10 and IFN-γ; 6) PSA changes;
S55, micro-invasion and microangiogenesis of implanted prostate tumor tissues were observed by electron microscopy; the expression of VGEF, PSCN, CD44+, CD31+, CD24+ in tumor tissues of each group was detected; the infiltration of inflammatory cells in pathological specimens was examined; changes in tumor microenvironment was investigated; and
S56, using ECT or PET-CT imaging method, observing the distribution of ¹²⁵I in mice after treatment; observing whether there are toxic side effects after application of ¹²⁵I-RSOAds-hTERT/PSA in tumor-bearing mice, and the applied dosage is obtained.

7. The experimental research method according to claim 2, wherein the step S3 specifically comprises the following steps,
S31, the ¹²⁵I label was labeled with N-bromosuccinimide (NBS) as an oxidant, and different concentrations of oncolytic virus and NBS were set to determine the optimal labeling conditions; the effect of the amount of ¹²⁵I, reaction time, pH and reaction volume on the labeling rate of ¹²⁵I-RSOAds-hTERT/PSA nuclides-oncolytic virus markers were investigated;
S32, after separation, the gel-column chromatography was used to separate and purify the radionuclide-oncolytic adenovirus marker; the radiochromic purity of ¹²⁵I-RSOAds-hTERT/PSA label was determined by paper chromatography at different times, using microporous membrane filter and sterilize, and storing the marker in a 4° C. refrigerator for use; and
S33, ¹²⁵I was labeled with dual-regulated oncolytic adenovirus to complete the pre-experiment, and the optimal labeling method, labeling conditions, labeling rate, and external conditions comprising temperature, time and pH were used to influence the success of the labeling.

8. The experimental research method according to claim 3, wherein the step S3 specifically comprises the following steps,
S31, the ¹²⁵I label was labeled with N-bromosuccinimide (NBS) as an oxidant, and different concentrations of oncolytic virus and NBS were set to determine the optimal labeling conditions; the effect of the amount of ¹²⁵I, reaction time, pH and reaction volume on the labeling rate of ¹²⁵I-RSOAds-hTERT/PSA nuclides-oncolytic virus markers were investigated;
S32, after separation, the gel-column chromatography was used to separate and purify the radionuclide-oncolytic adenovirus marker; the radiochromic purity of ¹²⁵I-RSOAds-hTERT/PSA label was determined by paper chromatography at different times, using microporous membrane filter and sterilize, and storing the marker in a 4° C. refrigerator for use; and
S33, ¹²⁵I was labeled with dual-regulated oncolytic adenovirus to complete the pre-experiment, and the optimal labeling method, labeling conditions, labeling rate, and external conditions comprising temperature, time and pH were used to influence the success of the labeling.

9. The experimental research method according to claim 2, wherein the step S4 specifically comprises the following steps,
S41, RT-PCR and Western blotting were used to identify two prostate cancer cell lines, comprising the human androgen-independent prostate cancer cell line PC3, the mouse androgen-independent prostate cancer cell line RM-1 and normal prostate tissue. Biological activity expression of hTERT/PSA;
S42, cultivating various types of prostate cancer cells with different expression levels of hTERT/PSA, and detecting expression of surface molecules such as prostate cancer stem cell antigens PSCN, CD44+, CD24+ in each group of cells;
S43, in vitro experiments were divided into 4 groups: virus-nuclear complex group (125I-RSOAds-hTERT-PSA), nuclear-free RSOAds-hTERT-PSA group, simple radionuclide group 125I, saline blank control group, according to the experiment requires the addition of two types of in vitro cultured prostate cancer cells in each experimental group, and compares the killing effects of the above four groups on prostate cancer cells with different hTERT and PSA expression;
S44, each group was tested as follows and repeated at least 3 times: a detailed statistical analysis of the collected data was performed to observe the killing effect of 125I-RSOAds-hTERT/PSA on prostate cancer cell growth:
detection of the expression of the oncolytic adenovirus E1A/E1B gene in ¹²⁵I-RSOAds-hTERT/PSA; detection of oncolytic adenovirus replication in ¹²⁵I-RSOAds-hTERT/PSA; determination of ¹²⁵I-RSOAds-hTERT/PSA on prostate cancer cells killing effect; ELISA was used to detect the secretion of cytokines in the supernatant of each group, and the changes of immune indexes were observed; the apoptosis of prostate cancer cells in each group was detected by TUNNEL method and flow cytometry, and some specific apoptosis induction index was detected; expression of prostate cancer stem cell antigens PSCN, CD44+, CD24+ in prostate cancer tumor cells was detected; and
S45, the concentration of nuclide ¹²⁵I in prostate cancer cells was examined; the dose of nuclide ¹²⁵I was measured by the same killing effect, and the stability of nuclide ¹²⁵I was examined during different culture periods.

10. The experimental research method according to claim 3, wherein the step S4 specifically comprises the following steps,
S41, RT-PCR and Western blotting were used to identify two prostate cancer cell lines, comprising the human androgen-independent prostate cancer cell line PC3, the mouse androgen-independent prostate cancer cell line RM-1 and normal prostate tissue. Biological activity expression of hTERT/PSA;
S42, cultivating various types of prostate cancer cells with different expression levels of hTERT/PSA, and detecting expression of surface molecules such as prostate cancer stem cell antigens PSCN, CD44+, CD24+ in each group of cells;
S43, in vitro experiments were divided into 4 groups: virus-nuclear complex group (125I-RSOAds-hTERT-PSA), nuclear-free RSOAds-hTERT-PSA group, simple radionuclide group 125I, saline blank control group, according to the experiment requires the addition of two types of in vitro cultured prostate cancer cells in each experimental group, and compares the killing effects of the above four groups on prostate cancer cells with different hTERT and PSA expression;
S44, each group was tested as follows and repeated at least 3 times: a detailed statistical analysis of the collected data was performed to observe the killing effect of 125I-RSOAds-hTERT/PSA on prostate cancer cell growth:
detection of the expression of the oncolytic adenovirus E1A/E1B gene in ¹²⁵I-RSOAds-hTERT/PSA; detection of oncolytic adenovirus replication in ¹²⁵I-RSOAds-hTERT/PSA; determination of ¹²⁵I-RSOAds-hTERT/PSA on prostate cancer cells killing effect; ELISA was used to detect the secretion of cytokines in the supernatant of each group, and the changes of immune indexes were observed; the apoptosis of prostate cancer cells in each group was detected by TUNNEL method and flow cytometry, and some specific apoptosis induction index was detected; expression of prostate cancer stem cell antigens PSCN, CD44+, CD24+ in prostate cancer tumor cells was detected; and
S45, the concentration of nuclide ¹²⁵I in prostate cancer cells was examined; the dose of nuclide ¹²⁵I was measured by the same killing effect, and the stability of nuclide ¹²⁵I was examined during different culture periods.

11. The experimental research method according to claim 2, wherein the step S5 specifically comprises the following steps,
S51, distribution of nuclear-virus complexes in normal mice: ¹²⁵I-RSOAds-hTERT-PSA was injected from the tail vein of mice, and ECT or PET-CT imaging was used at different time periods to determine the standard maximum intake value of different organ nuclide;
S52, establishing an implanted inbred C57BL/6 mouse prostate cancer animal model with reference to international and domestic literature: preparing a cell suspension of mouse androgen-independent prostate adenocarcinoma cell line RM-1 in log phase in vitro, and 10×10⁶ cells/mouse was injected subcutaneously into the right forelimb of the mouse or other suitable parts; the microscopic ultrasound and the touch method were used to observe the tumor formation; the experiment was performed when the tumor was about 2 g; PSA and some cytokines were examined by blood sampling from the tail vein;
S53, animal experiments were randomly divided into 4 groups (n=20): radionuclide-virus (¹²⁵I-RSOAds-hTERT-PSA) marker group, unlabeled radionuclide RSOAds-hTERT-PSA group, simple nuclear group ¹²⁵I group, normal saline in the blank control group; mice in each experimental group were treated with direct injection of prostate cancer and intravenous administration of mice, and anti-tumor effect of the ¹²⁵I-RSOAds-hTERT-PSA marker, the double-regulated oncolytic adenovirus RSOAds-hTERT-PSA, and the application of radionuclide ¹²⁵I were compared;
S54, each experimental group performs the following observation and detection in different time periods:
1) tumor growth curve, survival observation and transplanted tumor volume of tumor-bearing mice (using micro-ultrasound); 2) detection of tumor tissue transfected adenovirus E1A/E1B protein content, study transfection efficiency, observe ¹²⁵I-RSOAds-hTERT-PSA can directly target prostate cancer cells; 3) prostate cancer cell apoptosis detection (TUNNEL method and flow cytometry); Western Blot detection of Caspase-3 and other expression levels, to explore apoptosis-induced pathways; 4) prostate cancer transplanted tumors and pathological examination of important organs (HE staining, immunohistochemistry), examination of CD4+, CD8+ T cells and macrophage infiltration in the tumor; 5) ELISA method to detect the secretion of cytokines in the serum of mice comprising IL-2, TNF, IL-10 and IFN-γ; 6) PSA changes;
S55, micro-invasion and microangiogenesis of implanted prostate tumor tissues were observed by electron microscopy; the expression of VGEF, PSCN, CD44+, CD31+, CD24+ in tumor tissues of each group was detected; the infiltration of inflammatory cells in pathological specimens was examined; changes in tumor microenvironment was investigated; and S56, using ECT or PET-CT imaging method, observing the distribution of ¹²⁵I in mice after treatment; observing whether there are toxic side effects after application of ¹²⁵I-RSOAds-hTERT/PSA in tumor-bearing mice, and the applied dosage is obtained.

12. The experimental research method according to claim 3, wherein the step S5 specifically comprises the following steps,
S51, distribution of nuclear-virus complexes in normal mice: ¹²⁵I-RSOAds-hTERT-PSA was injected from the tail vein of mice, and ECT or PET-CT imaging was used at different time periods to determine the standard maximum intake value of different organ nuclide;
S52, establishing an implanted inbred C57BL/6 mouse prostate cancer animal model with reference to international and domestic literature: preparing a cell suspension of mouse androgen-independent prostate adenocarcinoma cell line RM-1 in log phase in vitro, and 10×10⁶ cells/mouse was injected subcutaneously into the right forelimb of the mouse or other suitable parts; the microscopic ultrasound and the touch method were used to observe the tumor formation; the experiment was performed when the tumor was about 2 g; PSA and some cytokines were examined by blood sampling from the tail vein;
S53, animal experiments were randomly divided into 4 groups (n=20): radionuclide-virus (¹²⁵I-RSOAds-hTERT-PSA) marker group, unlabeled radionuclide RSOAds-hTERT-PSA group, simple nuclear group ¹²⁵I group, normal saline in the blank control group; mice in each experimental group were treated with direct injection of prostate cancer and intravenous administration of mice, and anti-tumor effect of the ¹²⁵I-RSOAds-hTERT-PSA marker, the double-regulated oncolytic adenovirus RSOAds-hTERT-PSA, and the application of radionuclide ¹²⁵I were compared;
S54, each experimental group performs the following observation and detection in different time periods:
1) tumor growth curve, survival observation and transplanted tumor volume of tumor-bearing mice (using micro-ultrasound); 2) detection of tumor tissue transfected adenovirus E1A/E1B protein content, study transfection efficiency, observe ¹²⁵I-RSOAds-hTERT-PSA can directly target prostate cancer cells; 3) prostate cancer cell apoptosis detection (TUNNEL method and flow cytometry); Western Blot detection of Caspase-3 and other expression levels, to explore apoptosis-induced pathways; 4) prostate cancer transplanted tumors and pathological examination of important organs (HE staining, immunohistochemistry), examination of CD4+, CD8+ T cells and macrophage infiltration in the tumor; 5) ELISA method to detect the secretion of cytokines in the serum of mice comprising IL-2, TNF, IL-10 and IFN-γ; 6) PSA changes;
S55, micro-invasion and microangiogenesis of implanted prostate tumor tissues were observed by electron microscopy; the expression of VGEF, PSCN, CD44+, CD31+, CD24+ in tumor tissues of each group was detected; the infiltration of inflammatory cells in pathological specimens was examined; changes in tumor microenvironment was investigated; and
S56, using ECT or PET-CT imaging method, observing the distribution of ¹²⁵I in mice after treatment; observing whether there are toxic side effects after application of ¹²⁵I-RSOAds-hTERT/PSA in tumor-bearing mice, and the applied dosage is obtained.
